(19) United States
(12) Patent Application Publication
(10) Pub. No.: US 2012/0039748 A1
(43) Pub. Date: Feb. 16, 2012

(54) AUTOMATIC ANALYSIS APPARATUS

(75) Inventors: Tomonori Mimura, Kasama (JP); Akihisa Makino, Hitachinaka (JP); Sakuichiro Adachi, Kawasaki (JP)

(73) Assignee: HITACHI HIGH-TECHNOLOGIES CORPORATION, Tokyo (JP)

(21) Appl. No.: 13/202,337

(22) PCT Filed: Feb. 24, 2010
(86) PCT No.: PCT/JP2010/052868

§ 371 Date: Aug. 19, 2011

(30) Foreign Application Priority Data
Mar. 18, 2009 (JP) 2009-065393

(51) Int. Cl. G01N 33/00

(52) U.S. Cl. 422/63

(57) ABSTRACT

An automatic analysis apparatus capable of testing a plurality of items with high accuracy and at low cost without increasing the size of the apparatus is provided. An automatic analysis apparatus 1 having test mechanisms for a plurality of items including a photometric mechanism 70 and flow analysis mechanisms 40a and 40b is provided with a common disk (disk for reaction and pretreatment) 20. In the common disk 20, fixed containers 21 fixed to the common disk 20 and detachably-provided disposable containers 22 are disposed. Therefore, while achieving the downsizing of the apparatus, the containers to be used can be selected in accordance with the test items in such a manner that the fixed containers 21 are used as high-accuracy containers to be cleaned and reused and the disposable containers 22 are used as low-cost disposable containers.

### TECHNICAL FIELD

The present invention relates to an automatic analysis apparatus that automatically analyzes components of blood and others, and more particularly relates to techniques effectively applied to an apparatus which carries out a plurality of test items such as a biochemical test, an immunoserological test and a blood coagulation test.

### BACKGROUND ART

Conventionally, the efficiency of clinical test has been bad because tests such as an immunoserological test and a blood coagulation test are separately carried out in respectively dedicated apparatuses and it has been required to carry out a plurality of tasks such as moving specimens between the apparatuses, setting the specimens to the apparatuses, operations for requesting analysis, and summarizing output results. Therefore, laborsaving of the tests and speeding-up of test reports by carrying out the series of tests more efficiently and downsizing by integration of the test apparatuses have been desired.

There is known an apparatus configured to be able to carry out a plurality of test items such as a biochemical test, an immunoserological test and a blood coagulation test in the same apparatus in consideration of these demands. Also in such an apparatus, proposals for further simplifying the configuration thereof have been made, and for example, Patent Document 1 has proposed an apparatus made up of a first test mechanism which carries out biochemical and immunoserological tests and a second test mechanism which carries out an immunological coagulation test. Patent Document 2 has proposed a test method in which many reaction containers are sequentially moved by a container-by-container moving operation and light intensity is calculated in reciprocating operation at the photometric intervals in accordance with each sample.

Moreover, in the clinical test, various pretreatments such as specimen (sample) dilution, hemolysis of HbA1c and B/F separation of antigen-antibody reactions in an immunoserological test are required. Since the pretreatment is carried out in a dedicated machine of each test in many cases, in order to carry out pretreatment and analysis in one apparatus, mutually-independent dedicated machines are connected by a specimen rack conveyance line and operated like in, for example, a modular method. The modular method can connect various dedicated machines almost freely and is excellent in terms of readiness to handle analysis varieties and processing ability, but on the other hand, the method has problems such as increase in the size of the apparatus and increase in the apparatus price.

Therefore, a pretreatment disk which commonly carries out the pretreatments of the tests is convenient for carrying out such various analyses in one apparatus. However, if reaction disks for respective test items are present in addition to the pretreatment disk, there is a problem that the size of the apparatus is increased.

In consideration of such problems, recently, an apparatus provided with a multi-purpose disk which can not only carry out a plurality of test items but also carry out the pretreatment with a reaction disk for performing the reactions between samples and reagents is commercially sold.

### PRIOR ART DOCUMENTS

### Patent Documents

Patent Document 1: Japanese Unexamined Patent Application Publication No. 2001-13151
Patent Document 2: Japanese Unexamined Patent Application Publication No. 2001-27639

### SUMMARY OF THE INVENTION

### Problems to be Solved by the Invention

However, in both of the proposals of Patent Document 1 and Patent Document 2, the pretreatment is carried out by a different apparatus, and the apparatus configuration including the pretreatment cannot be simplified.

Also, in the above-described apparatus provided with the multi-purpose disk which is recently commercially sold, disposable containers are used in both a single-use fashion and a reusing fashion. Therefore, when used in a reusing fashion, there is a problem that the surface accuracy of a light projection surface in highly accurate colorimetric analysis becomes insufficient, and when used in a single-use fashion, there is a problem that cost is increased by the degree corresponding to the processing performed for improving the surface accuracy so that the container can be reused.

Herein, although there is an apparatus which carries out the pretreatment with a reused reaction container of a reaction disk for a biochemical test, the apparatus cannot handle a plurality of test items.

Therefore, it has been desired that a multi-purpose disk of an apparatus which handles a plurality of test items is provided with high-accuracy containers to be cleaned and reused and low-cost disposable containers.

The present invention has been made in view of the above-described problems, and an object of the present invention is to provide an automatic analysis apparatus capable of testing a plurality of items with high accuracy and at low cost without increasing the size of the apparatus.

The above and other objects and novel characteristics of the present invention will be apparent from the description of the present specification and the accompanying drawings.

### Means for Solving the Problems

The following is a brief description of an outline of the typical invention disclosed in the present application.

An automatic analysis apparatus according to the present invention is an automatic analysis apparatus including: containers for carrying out a reaction between a sample and a reagent and/or pretreatment of the sample by pretreatment liquid; a disk for the reaction and the pretreatment, on which the containers are disposed; a sample dispensing mechanism for dispensing the sample into the container; and a test mechanism for carrying out a plurality of items based on the reaction, and a fixed container fixed to the disk for the reaction and the pretreatment and a disposable container detachably provided on the disk for the reaction and the pretreatment are disposed as the containers on the disk for the reaction and the pretreatment.

### Effects of the Invention

The effects obtained by typical embodiments of the invention disclosed in the present application will be briefly described below.

That is, since fixed containers and detachably-provided disposable containers are disposed on a disk for reaction and pretreatment of an automatic analysis apparatus having mechanisms for carrying out a plurality of test items, the containers to be used can be selected in accordance with the test items in such a manner that the fixed containers are used as high-accuracy containers to be cleaned and reused and the disposable containers are used as low-cost disposable containers. Accordingly, the plurality of test items can be carried out with high accuracy and at low cost without increasing the size of the apparatus.

### BRIEF DESCRIPTIONS OF THE DRAWINGS

FIG. 1 is a schematic plan view showing an outline of an embodiment of an automatic analysis apparatus of the present invention;

FIG. 2 is a drawing showing the flows of samples, pretreatment liquid, reagents and disposable containers in the automatic analysis apparatus of FIG. 1;

FIG. 3A is an explanatory drawing for describing the flows of operations in the automatic analysis apparatus of FIG. 1;

FIG. 3B is an explanatory drawing for describing the flows of operations in the automatic analysis apparatus of FIG. 1;

FIG. 3C is an explanatory drawing for describing the flows of operations in the automatic analysis apparatus of FIG. 1;

FIG. 4A is an explanatory drawing for describing the flows of operations in the automatic analysis apparatus of FIG. 1;

FIG. 4B is an explanatory drawing for describing the flows of operations in the automatic analysis apparatus of FIG. 1;

FIG. 4C is an explanatory drawing for describing the flows of operations in the automatic analysis apparatus of FIG. 1;

FIG. 5A is an explanatory drawing for describing the flows of operations in the automatic analysis apparatus of FIG. 1;

FIG. 5B is an explanatory drawing for describing the flows of operations in the automatic analysis apparatus of FIG. 1;

FIG. 5C is an explanatory drawing for describing the flows of operations in the automatic analysis apparatus of FIG. 1;

FIG. 6 is a schematic plan view showing an outline of an embodiment of an automatic analysis apparatus to which a different sample dispensing mechanism is applied;

FIG. 7 is an explanatory drawing for describing a basic cycle of a common disk;

FIG. 8 is an explanatory drawing for describing a rotating operation of the common disk in A-cycles;

FIG. 9A is an explanatory drawing for describing rotating operations of the common disk in B-cycles;

FIG. 9B is an explanatory drawing for describing rotating operations of the common disk in B-cycles;

FIG. 10A is an explanatory drawing for describing an example of operations in the case of the shortest cycle with the basic cycle of FIG. 7;

FIG. 10B is an explanatory drawing for describing an example of operations in the case of the shortest cycle with the basic cycle of FIG. 7;

FIG. 10C is an explanatory drawing for describing an example of operations in the case of the shortest cycle with the basic cycle of FIG. 7;

FIG. 10D is an explanatory drawing for describing an example of operations in the case of the shortest cycle with the basic cycle of FIG. 7;

FIG. 11 is a drawing showing an example of the rotating operation of the common disk in the case in which 20 containers are disposed;

FIG. 12 is a drawing showing an example of the rotating operation of the common disk in the case in which 20 containers are disposed;

FIG. 13 is a drawing showing an example of the rotating operation of the common disk in the case in which 20 containers are disposed;

FIG. 14A is a main-part side view showing a main part of a sample dispensing probe in the case in which it is used with cleaning;

FIG. 14B is a main-part side view showing a main part of the sample dispensing probe in the case in which a disposable chip is used;

FIG. 15A is an explanatory drawing for describing the flow of the operations of the sample dispensing probe in the case in which it is used with cleaning;

FIG. 15B is an explanatory drawing for describing the flow of the operations of the sample dispensing probe in the case in which it is used with cleaning;

FIG. 15C is an explanatory drawing for describing the flow of the operations of the sample dispensing probe in the case in which it is used with cleaning;

FIG. 15D is an explanatory drawing for describing the flow of the operations of the sample dispensing probe in the case in which it is used with cleaning;

FIG. 15E is an explanatory drawing for describing the flow of the operations of the sample dispensing probe in the case in which it is used with cleaning;

FIG. 15F is an explanatory drawing for describing the flow of the operations of the sample dispensing probe in the case in which it is used with cleaning;

FIG. 15G is an explanatory drawing for describing the flow of the operations of the sample dispensing probe in the case in which it is used with cleaning;

FIG. 15H is an explanatory drawing for describing the flow of the operations of the sample dispensing probe in the case in which it is used with cleaning;

FIG. 16A is an explanatory drawing for describing the flow of the operations of the sample dispensing probe in the case in which the disposable chip is used;

FIG. 16B is an explanatory drawing for describing the flow of the operations of the sample dispensing probe in the case in which the disposable chip is used;

FIG. 16C is an explanatory drawing for describing the flow of the operations of the sample dispensing probe in the case in which the disposable chip is used;

FIG. 16D is an explanatory drawing for describing the flow of the operations of the sample dispensing probe in the case in which the disposable chip is used;

FIG. 16E is an explanatory drawing for describing the flow of the operations of the sample dispensing probe in the case in which the disposable chip is used;

FIG. 16F is an explanatory drawing for describing the flow of the operations of the sample dispensing probe in the case in which the disposable chip is used;

FIG. 16G is an explanatory drawing for describing the flow of the operations of the sample dispensing probe in the case in which the disposable chip is used;

FIG. 16H is an explanatory drawing for describing the flow of the operations of the sample dispensing probe in the case in which the disposable chip is used;

FIG. 17A is an explanatory drawing for describing the flow of the operations of the sample dispensing probe in the case in which the disposable chip is used;

FIG. 17B is an explanatory drawing for describing the flow of the operations of the sample dispensing probe in the case in which the disposable chip is used; and

FIG. 17C is an explanatory drawing for describing the flow of the operations of the sample dispensing probe in the case in which the disposable chip is used.

### BEST MODE FOR CARRYING OUT THE INVENTION

Hereinafter, embodiments of the present invention will be described in detail with reference to the accompanying drawings. Note that components having the same function are denoted by the same reference symbols throughout the drawings for describing the embodiments, and the repetitive description thereof will be omitted as much as possible.

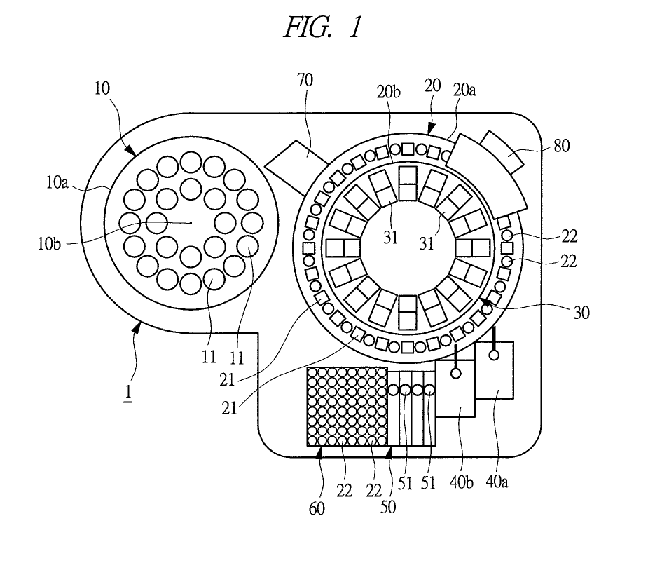

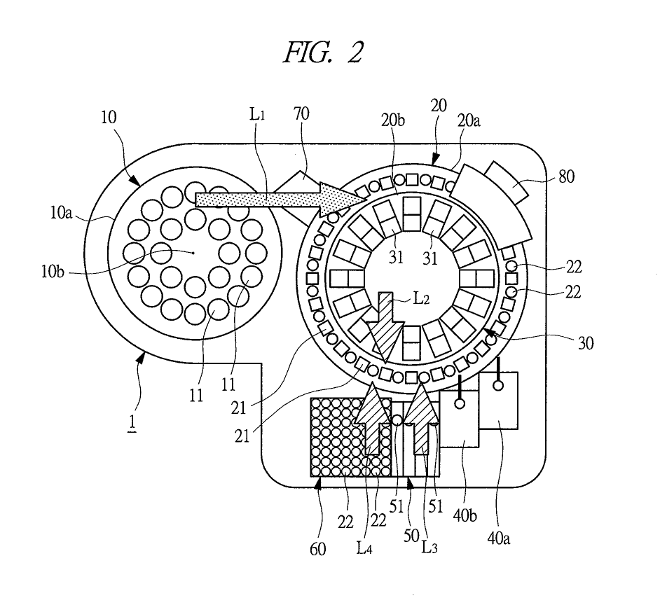

FIG. 1 is a schematic plan view showing an outline of an embodiment of an automatic analysis apparatus of the present invention, and FIG. 2 is a drawing showing the flows of samples, pretreatment liquid, reagents, and disposable containers in the automatic analysis apparatus of FIG. 1. In FIG. 1 and FIG. 2, illustration of sample dispensing mechanisms is intentionally omitted in order to facilitate understanding. Also, illustration of a control unit, a display unit, an input unit and a memory unit is also intentionally omitted in all of the drawings.

As shown in FIG. 1, the automatic analysis apparatus 1 is provided with: a sample disk 10; a common disk (disk for reaction and pretreatment) 20; a biochemical-test reagent disk 30; flow analysis mechanisms 40a and 40b serving as mechanisms for carrying out a plurality of test items; a pretreatment liquid and flow analysis reagent container storage unit (hereinafter, pretreatment liquid container storage unit) 50; and a disposable container storage unit 60.

In the sample disk 10, sample containers 11 retaining samples are disposed on the outer circumferential 10a side and on the center 10b side at predetermined intervals.

The common disk 20 is provided on a lateral side of the sample disk 10 and is configured to carry out both the reactions between samples and reagents and the pretreatment of the samples by the pretreatment liquid. Note that “pretreatment” described in the present invention includes dilution of a sample.

The common disk 20 is provided with, on the outer circumferential 20a side, a photometric mechanism (test mechanism) 70 for biochemical tests serving as one of test mechanisms for a plurality of items, a container cleaning mechanism 80, and though not shown in the drawing, an agitating mechanism which agitates the sample with the pretreatment liquid or the reagent. The photometric mechanism 70 includes a light source which delivers analysis light for analyzing the reaction liquid in the containers, a detector which disperses and detects the analysis light transmitted through the reaction liquid and others though not shown. The photometric mechanism 70 may be used for the test items other than those of biochemical.

Along the circumferential direction of the common disk 20, fixed containers 21 fixed to the common disk 20 and disposable containers 22 which are detachably provided are alternately disposed. The fixed containers 21 are reused through cleaning by the container cleaning mechanism 80, and the disposable containers 22 are discarded after use.

Publicly-known containers having high surface accuracy so as to support the calorimetric analysis can be used as the fixed containers 21, and publicly-known containers can also be used as the disposable containers 22. In the example shown in the drawings, the fixed containers 21 and the disposable containers 22 are alternately disposed from the viewpoint for facilitating control. However, they are only required to be regularly disposed, for example, the disposable container 22 is disposed for every two containers. Also, the number of the disposed containers can be arbitrarily increased/decreased in accordance with needs.

On the inner circumferential 20b side of the common disk 20, the biochemical-test reagent disk 30 is provided. In the biochemical-test reagent disk 30, biochemical-test reagent cassettes 31 each of which retains a first reagent and a second reagent for biochemical tests in one container are disposed along the circumferential direction. By virtue of this, the need for separately storing the first reagent and the second reagent is eliminated. However, the first reagent and the second reagent may be separately retained by containers such as test tubes. Also, the biochemical-test reagent disk 30 may be provided at a different position in the automatic analysis apparatus 1. Furthermore, the biochemical-test reagent disk 30 and the biochemical-test reagent cassettes 31 may retain a reagent for a different test item.

The flow analysis mechanisms 40a and 40b, the pretreatment liquid container storage unit 50 and the disposable container storage unit 60 are provided on the front side of the apparatus compared with the common disk 20.

The flow analysis mechanisms 40a and 40b are capable of arbitrarily selecting and carrying out publicly-known tests as flow analysis such as an immunoserological test, a blood coagulation test, and an electrolyte test in accordance with requested items. The number of the flow analysis mechanisms may be arbitrarily increased, reduced or eliminated in accordance with requested items, and by this means, the apparatus can be further downsized.

As a matter of course, a container 51 stored in the pretreatment liquid container storage unit 50 retains pretreatment liquid when pretreatment is to be carried out and retains a corresponding flow analysis reagent when flow analysis is to be carried out.

The disposable containers 22 stored in the disposable container storage unit 60 can be arbitrarily exchanged with the disposable containers 22 of the common disk 20 by a disposable container transporting mechanism (not shown in FIG. 1).

As described above, in the automatic analysis apparatus 1, as shown by arrows L₁ to L₄ shown in FIG. 2, the flows of dispensing the samples, reagents and pretreatment liquid from the containers of the sample disk 10, the biochemical-test reagent disk 30 and the pretreatment liquid container storage unit 50 to the fixed containers 21 or the disposable containers 22 of the common disk 20 are formed. Moreover, the flow of transportation of the disposable containers 22 from the disposable container storage unit 60 to the common disk 20 is formed.

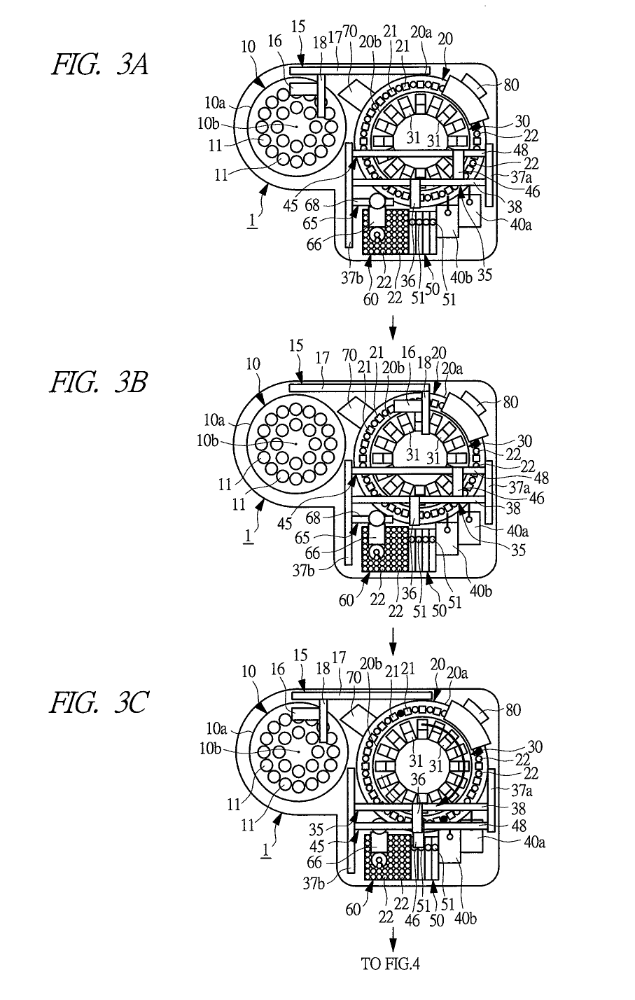

Subsequently, the flow of the operation of the automatic analysis apparatus 1 will be described with taking the case in which pretreatment is required in a biochemical test as an example. FIGS. 3A to 3C, FIGS. 4A to 4C, and FIGS. 5A to 5C are explanatory drawings for describing the flow of the operation in the automatic analysis apparatus of FIG. 1 in this order.

First of all, as shown in FIG. 3A, the actual automatic analysis apparatus 1 is further provided with a sample dispensing mechanism 15, a first reagent dispensing mechanism 35, a second reagent dispensing mechanism 45 and a disposable container transporting mechanism 65.

The sample dispensing mechanism 15 is a so-called XY-rail type dispensing mechanism provided with a sample dispensing arm 16, a horizontal rail (first guide member) 17 provided along the width direction of the apparatus, in other words, the horizontal direction (first direction) and a perpendicular rail (second guide member) 18 provided along the depth direction of the apparatus (second direction orthogonal to the first direction). Therefore, the sample dispensing mechanism 15 is capable of arbitrarily approaching the container at any position on the common disk 20 and dispensing a sample thereat. Note that the state of being orthogonal to the horizontal direction is expressed as “perpendicular” herein.

The horizontal rail 17 is provided from the sample disk 10 to the common disk 20 in the rear end of the apparatus. The perpendicular rail 18 is attached so as to extend from the horizontal rail 17 toward the inner side of the apparatus and slides on the horizontal rail 17. The sample dispensing arm 16 is attached to the perpendicular rail 18 and slides on the perpendicular rail 18. In other words, the horizontal movement of the sample dispensing arm 16 is guided by the horizontal rail 17 via the perpendicular rail 18, and the depth-direction movement of the arm is directly guided by the perpendicular rail 18.

The first reagent dispensing mechanism 35, the second reagent dispensing mechanism 45 and the disposable container transporting mechanism 65 are all XY-rail type dispensing mechanisms like the sample dispensing mechanism 15.

The first reagent dispensing mechanism 35 is made up of a first reagent dispensing arm 36, perpendicular rails 37a and 37b provided at both ends in the horizontal direction so as to interpose the common disk 20 therebetween and a horizontal rail 38 installed between the perpendicular rails 37a and 37b. The horizontal rail 38 slides on the perpendicular rails 37a and 37b, and the first reagent dispensing arm 36 slides on the horizontal rail 38. The perpendicular rail 37b is formed to be longer toward the front side of the apparatus than the perpendicular rail 37a because the rail 37b is shared with the disposable container transporting mechanism 65.

The second reagent dispensing mechanism 45 shares the perpendicular rails 37a and 37b with the first reagent dispensing mechanism 35 and is provided with a second reagent dispensing arm 46 and a horizontal rail 48 installed between the perpendicular rails 37a and 37b. Also, like the first reagent dispensing mechanism 35, the horizontal rail 48 slides on the perpendicular rails 37a and 37b, and the second reagent dispensing arm 46 slides on the horizontal rail 48. The second reagent dispensing mechanism 45 is configured to function also as a pretreatment liquid dispensing mechanism, and by this means, the apparatus can be further downsized.

The disposable container transporting mechanism 65 is provided with a container holding arm 66, which holds the disposable container 22 at the distal end thereof, and a horizontal rail 68 attached so as to extend from the perpendicular rail 37b. The horizontal rail 68 slides on the perpendicular rail 37b, and the container holding arm 66 slides on the horizontal rail 68.

In the automatic analysis apparatus 1 provided with these mechanisms, when a biochemical test which requires pretreatment is started, the sample dispensing arm 16 of the sample dispensing mechanism 15 is moved to above the sample container 11 of the sample disk 10 by the horizontal rail 17 and the perpendicular rail 18 and sucks a sample in the sample container 11.

After the sample suction, as shown in FIG. 3B, the sample dispensing arm 16 is moved to above the disposable container 22 of the common disk 20 and discharges the sample into the disposable container 22.

After the sample discharge, as shown in FIG. 3C, the common disk 20 rotates clockwise (see an arrow in the drawing) to move the sample (at the black-colored positions in the drawing, and both of the positions before the movement and after the movement are colored in black in order to facilitate understanding). Also, the second reagent dispensing arm 46 of the second reagent dispensing mechanism 45 is moved to above the container 51 of the pretreatment liquid container storage unit 50 by the perpendicular rails 37a and 37b and the horizontal rail 48 and sucks pretreatment liquid in the container 51.

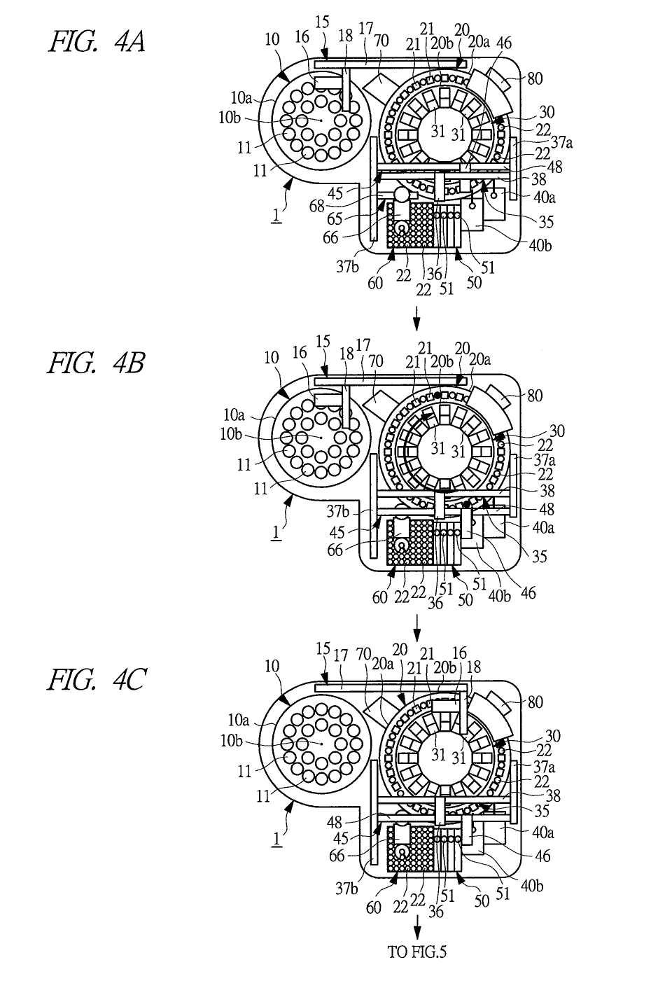

After the sample movement and the pretreatment liquid suction, as shown in FIG. 4A, the second reagent dispensing arm 46 is moved to above the disposable container 22 containing the sample of the common disk 20 and discharges the pretreatment liquid into the disposable container 22.

After the pretreatment liquid discharge, when the pretreatment is finished, as shown in FIG. 4B, the common disk 20 rotates clockwise (see an arrow in the drawing) to move the pretreated sample. In the example shown in the drawings, the second reagent dispensing mechanism 45 is moved toward the flow analysis mechanism 40a.

After the pretreated sample is moved, as shown in FIG. 4C, the sample dispensing arm 16 is moved to above the disposable container 22 containing the pretreated sample and sucks the pretreated sample in the disposable container 22.

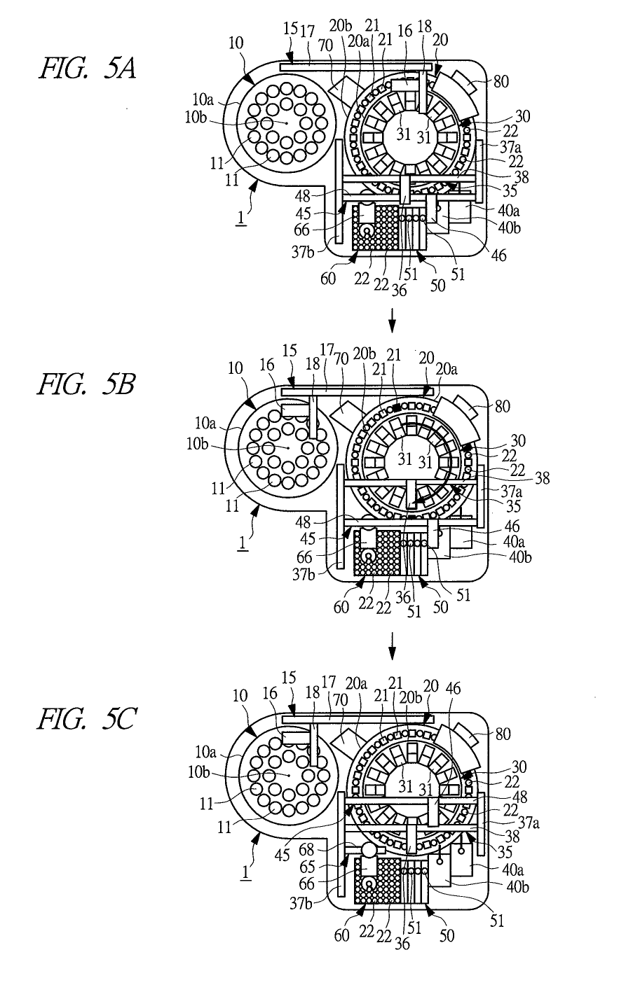

After the pretreated sample is sucked, as shown in FIG. 5A, the sample dispensing arm 16 is moved to above the fixed container 21, which is next to the disposable container 22 containing pretreated sample in the counterclockwise direction, and discharges the pretreated sample into the fixed container 21.

After the pretreated sample is discharged, as shown in FIG. 5B, the common disk 20 rotates clockwise (see an arrow in the drawing) to move the pretreated sample of the fixed container 21 (the black-colored positions in the drawing, and both of the positions before the movement and after the movement are colored in black in order to facilitate understanding).

Also, the first reagent dispensing arm 36 of the first reagent dispensing mechanism 35 is moved to above the biochemical-test reagent cassette 31 of the biochemical-test reagent disk 30 by the perpendicular rails 37a and 37b and the horizontal rail 38 and sucks the first reagent in the biochemical-test reagent cassette 31. The first reagent dispensing arm 36 sucks the first reagent from the biochemical-test reagent cassette 31 opposed to the fixed container 21 containing the pretreated sample via the edge of the inner circumference 20b of the common disk 20 in order to reduce the moving distance thereof.

After the pretreated sample movement and the first reagent suction, as shown in FIG. 5C, the first reagent dispensing arm 36 is moved to above the fixed container 21 containing the pretreated sample and discharges the first reagent into the fixed container 21. After the first reagent is discharged, as shown in the drawing, the second reagent dispensing arm 46 is moved to above the biochemical-test reagent cassette 31 in accordance with needs.

After the second reagent dispensing arm 46 is moved, though not shown, the arm sucks the second reagent, is then moved to above the fixed container 21 containing the pretreated sample and the first reagent, and discharges the second reagent into the fixed container 21. Note that the dispensing of the second reagent is carried out normally when about five minutes have elapsed after the discharge of the first reagent.

The reaction liquid after the above-described reaction between the sample and the first reagent and, in accordance with needs, the second reagent is moved to the photometric mechanism 70 by the rotation of the common disk 20, and the reaction liquid is analyzed. After the analysis is finished, the fixed container 21 is cleaned by the container cleaning mechanism 80. Meanwhile, the disposable container 22 is transported to the disposable container storage unit 60 by the container holding arm 66 of the disposable container transporting mechanism 65 and then discarded thereto.

In the case in which the pretreatment is sample dilution, there is no risk of contamination between samples, and therefore, the sample may be dispensed into the fixed container 21 from the beginning.

In the biochemical test that does not need pretreatment, operation approximately similar to that of the biochemical test that needs pretreatment is carried out except that the sample is discharged into the fixed container 21 instead of the disposable container 22 in FIG. 3B and the operations shown in FIG. 3C to FIG. 5A are not carried out.

The flow analysis in an immunoserological test and the like is similar to the biochemical test that needs pretreatment except that a flow reagent is dispensed instead of the pretreatment liquid in the operations of FIG. 3A to FIG. 4A. Then, after the reaction between the sample and the flow reagent, the reaction liquid is sucked by the flow analysis mechanisms 40a and 40b.

In the automatic analysis apparatus 1, the sample dispensing mechanism 15 may employ another publicly-known mechanism as long as the mechanism is capable of arbitrarily approaching the container at any position on the common disk 20 and dispensing the sample thereinto. FIG. 6 is a schematic plan view showing an outline of an embodiment of an automatic analysis apparatus to which another sample dispensing mechanism is applied.

The sample dispensing mechanism 25 shown in FIG. 6 is a multi-joint arm, which is provided with a rotation shaft 25a at the center of the common disk 20 and has a first joint 25b and a second joint 25c sequentially from the distal end thereof. More specifically, the sample dispensing mechanism 25 is capable of approaching an arbitrary container to dispense a sample because the first joint 25b is extended/contracted with using a joined part 25d between the first joint 25b and the second joint 25c as a rotation axis, and the second joint 25c rotates about the rotation shaft 25a. In the automatic analysis apparatus 1 of the present invention, it is preferred that the sample dispensing mechanism 15 shown in FIG. 1 to FIG. 5 and the sample dispensing mechanism 25 shown in FIG. 6 are applied from the viewpoint of control easiness.

In the example shown in the drawings, the second reagent dispensing mechanism 45 also serves as a pretreatment liquid dispensing mechanism in the automatic analysis apparatus 1. However, the automatic analysis apparatus 1 is not limited thereto, and the first reagent dispensing mechanism 35 may also serve as a pretreatment liquid dispensing mechanism, or both of the reagent dispensing mechanisms may also serve as pretreatment liquid dispensing mechanisms arbitrarily.

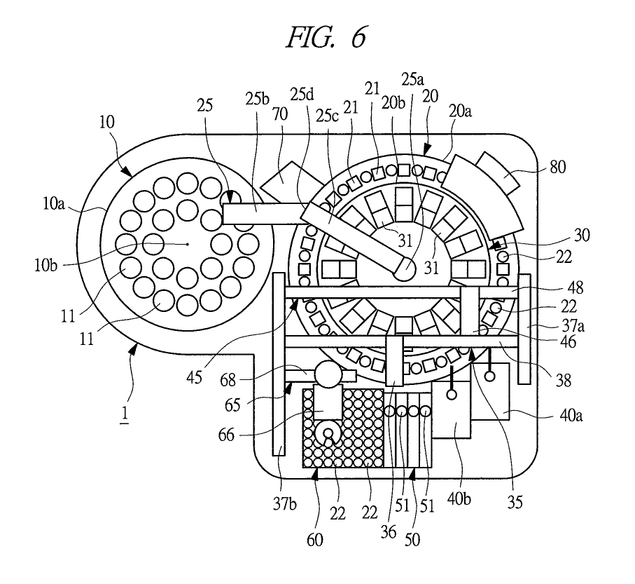

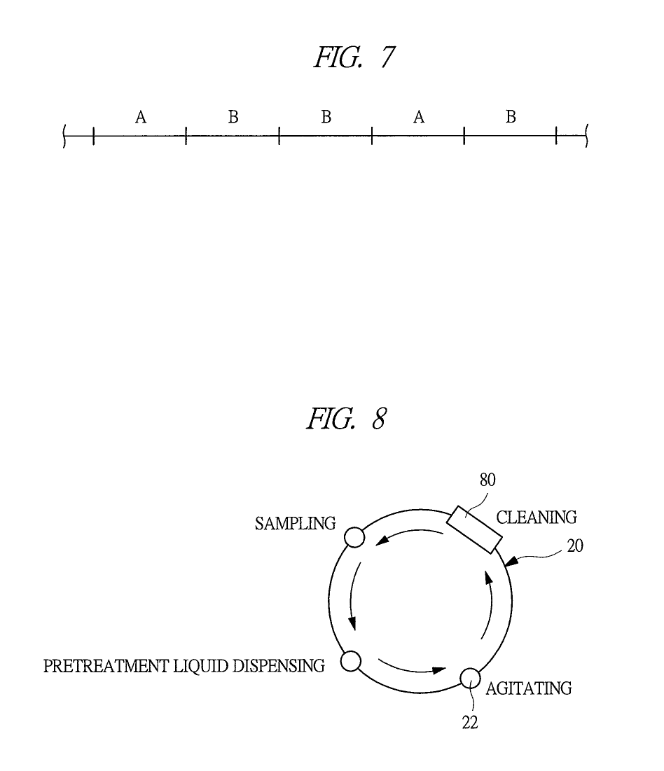

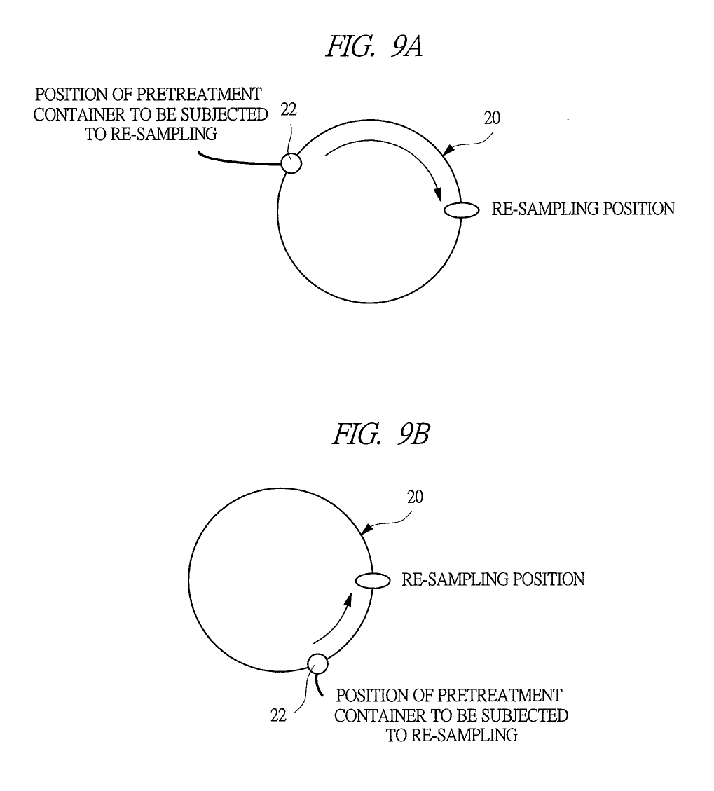

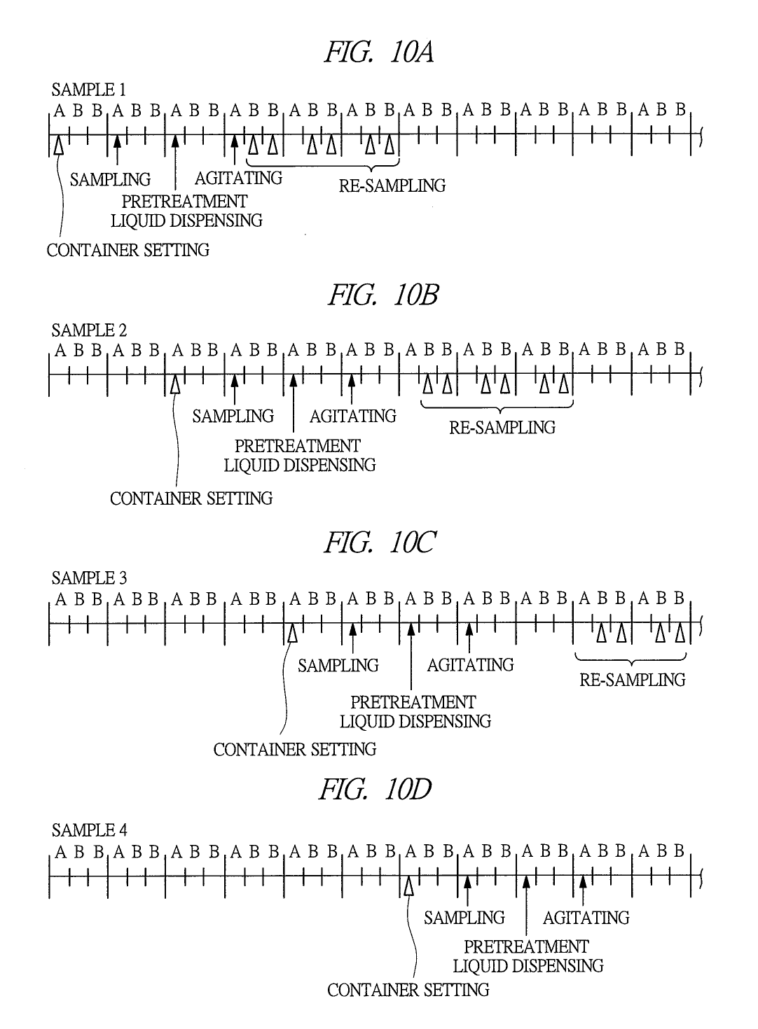

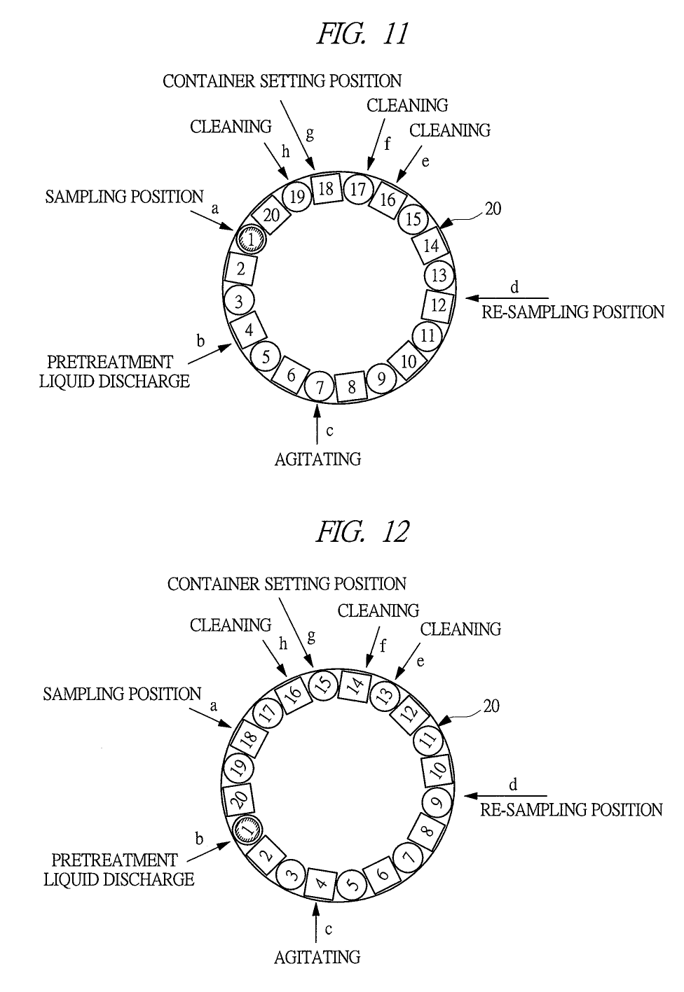

Subsequently, the rotating operation of the common disk 20 in the case in which pretreatment is carried out will be described. FIG. 7 is an explanatory drawing for describing a basic cycle of the common disk, FIG. 8 is an explanatory drawing for describing the rotating operation of the common disk in A-cycles, and FIGS. 9A and 9B are explanatory drawings for describing the rotating operation of the common disk in B-cycles. FIGS. 10A to 10D are explanatory drawings for describing an example of the operation in the case of the shortest cycle with the basic cycle of FIG. 7, and FIG. 11 to FIG. 13 are drawings showing examples of the rotating operation of the common disk in the case in which 20 containers are disposed. In FIG. 8, FIG. 9 and FIGS. 11 to 13, the disposition of the common disk is arbitrarily omitted or changed intentionally in order to facilitate understanding.

As shown in FIG. 7, in the operation of the common disk, the A-cycles of carrying out a pretreatment operation and the B-cycles of carrying out a re-sampling (pretreated sample dispensing) operation to an analysis unit are combined to provide the basic cycle. Each of the A-cycles and the B-cycles is independently controlled, but by making the cycle times thereof equal to each other, the operation timing of pretreatment and the operation timing to the analysis unit are standardized. In the example of FIG. 7, the two B-cycles are provided after the A-cycle, but the number of the B-cycles after the A-cycle may be arbitrarily changed in accordance with test items, the number of samples and others.

As shown in FIG. 8, in the A-cycles, each of sampling (sample dispensing), pretreatment liquid dispensing, agitating and cleaning is carried out in one cycle, respectively. The common disk 20 rotates regularly in one direction by a step serving as a common factor of the number obtained by adding one to the number of disposed containers such as that corresponding to N units of the disposable containers.

The B-cycle is operated at the point when the series of operations of the pretreatment up to the agitating in the A cycles is finished and the sample to be re-sampled is prepared. As shown in FIG. 9, in the B-cycle, no matter where the container to be subjected to re-sampling next is at any position on the common disk 20, the container is moved to a re-sampling position. The moving distance in this case is arbitrary, but the common disk 20 is configured to be able to select any of the clockwise rotation shown in FIG. 9A and the counterclockwise rotation shown in FIG. 9B so that the moving distance and time can be shortened.

In the case in which an untouched period or heating for a certain period of time is not required in the pretreatment, for example, the common disk is operated with respect to samples in the procedure as shown in FIGS. 10A to 10D.

First, as shown in FIG. 10A, in the first A-cycle, the disposable container for sampling a first sample (sample 1) is set, and in the next A-cycle, the sample 1 is sampled.

In the third A-cycle, the pretreatment liquid is dispensed into the sample 1, and as shown in FIG. 10B, the disposable container for sampling a sample 2 subsequent to the sample 1 is set. In the second A-cycle, the disposable container is not set because the fixed container is at a container setting position.

In the fourth A-cycle, the sample 1 is agitated, and the sample 2 is sampled. Also, the sample 1 is re-sampled in the two B-cycles subsequent to the A-cycle. Then, since there are six biochemical test items in the example shown in the drawings, re-sampling is carried out also in the two B-cycles of the next basic cycle and the basic cycle after the next basic cycle.

In the fifth A-cycle, the pretreatment liquid is dispensed into the sample 2, and as shown in FIG. 10C, the disposable container for sampling a sample 3 subsequent to the sample 2 is set. In the sixth A-cycle, the sample 2 is agitated, and the sample 3 is sampled. In the B-cycle subsequent to this A-cycle, re-sampling of the sample 1 is carried out, and therefore, re-sampling of the sample 2 is not carried out, but it is carried out in the B-cycles of the next and subsequent basic cycles.

In the seventh A-cycle, the pretreatment liquid is dispensed into the sample 3, and as shown in FIG. 10D, the disposable container for sampling a sample 4 subsequent to the sample 3 is set.

In this manner, sampling, pretreatment liquid dispensing, agitating and re-sampling are sequentially repeated. FIG. 10 shows the example of the case of the shortest cycle, and empty cycles may be arbitrarily provided in order to avoid overlapping or carry-over of the dispensing mechanisms.

For example, in the case in which a total of 20 fixed containers and disposable containers are disposed on the common disk 20, the common disk 20 is operated to rotate in the manner shown in FIG. 11 to FIG. 13. In FIG. 11 to FIG. 13, circular containers denoted by odd numbers represent the disposable containers, and rectangular containers denoted by even numbers represent the fixed containers.

First, as shown in FIG. 11, at the timing of the A-cycle, the common disk 20 rotates counterclockwise by the distance corresponding to three containers, and the disposable container 22 denoted by the number “1” set at the position of “g” is subjected to sampling at the position of “a”.

Next, as shown in FIG. 12, the common disk 20 further rotates counterclockwise by the distance corresponding to three containers, and the pretreatment liquid is dispensed thereinto at the position of “b”.

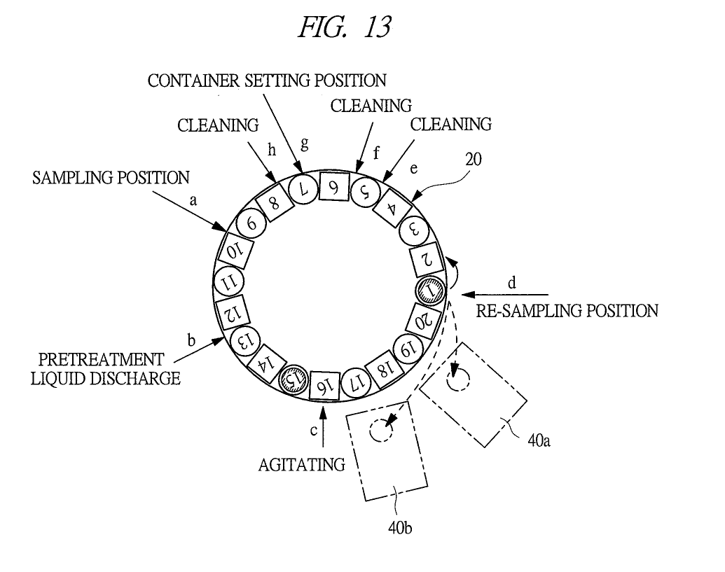

Then, the common disk 20 further rotates counterclockwise by the distance corresponding to three containers, and the pretreatment liquid is agitated at the position of “c”. Thereafter, as shown in FIG. 13, the common disk 20 rotates to the position of “d” at the timing of the B-cycle, and at this position, re-sampling from the disposable container denoted by the number “1” to the next fixed container denoted by the number “2” is carried out. At this time, in the flow analysis, the flow reagent is dispensed at the position of “b”, and re-sampling to the flow analysis mechanisms 40a and 40b is carried out at the position of “d”. Note that, in the state of FIG. 13, a sample has been already dispensed also into the disposable container denoted by the number “15”.

After the re-sampling is finished, again at the timing of the next A-cycle, the fixed container is subjected to sample suction, cleaning liquid discharge and cleaning sequentially at the positions of “e”, “f” and “h” of the cleaning mechanism, and the disposable container is discarded.

Incidentally, the sample dispensing arm 16 of the above-described sample dispensing mechanism 15 retains a sample dispensing probe. The sample dispensing probe is configured to allow both the use with cleaning and the use of a disposable chip.

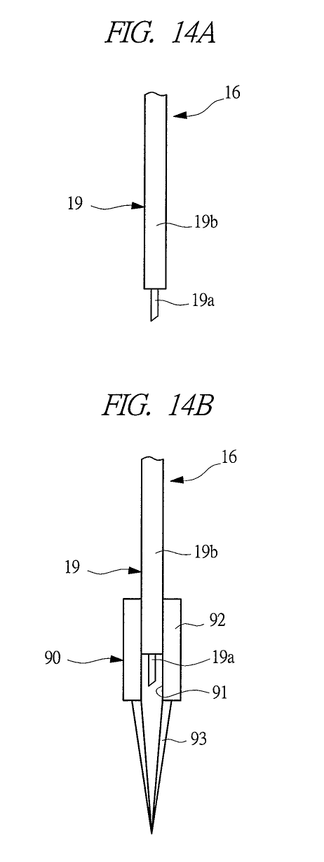

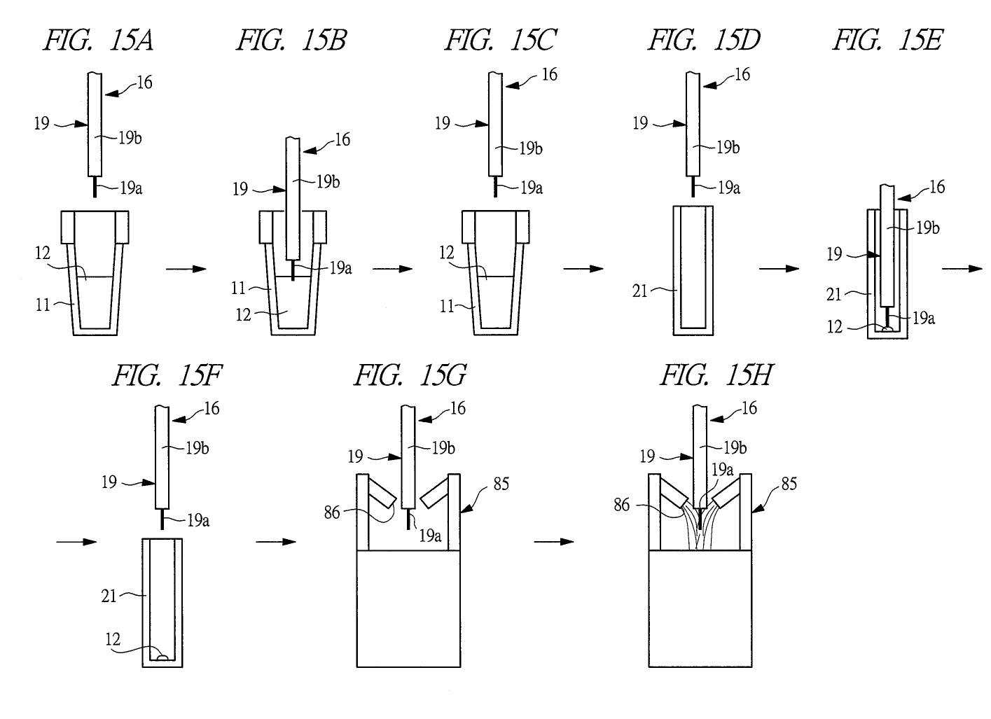

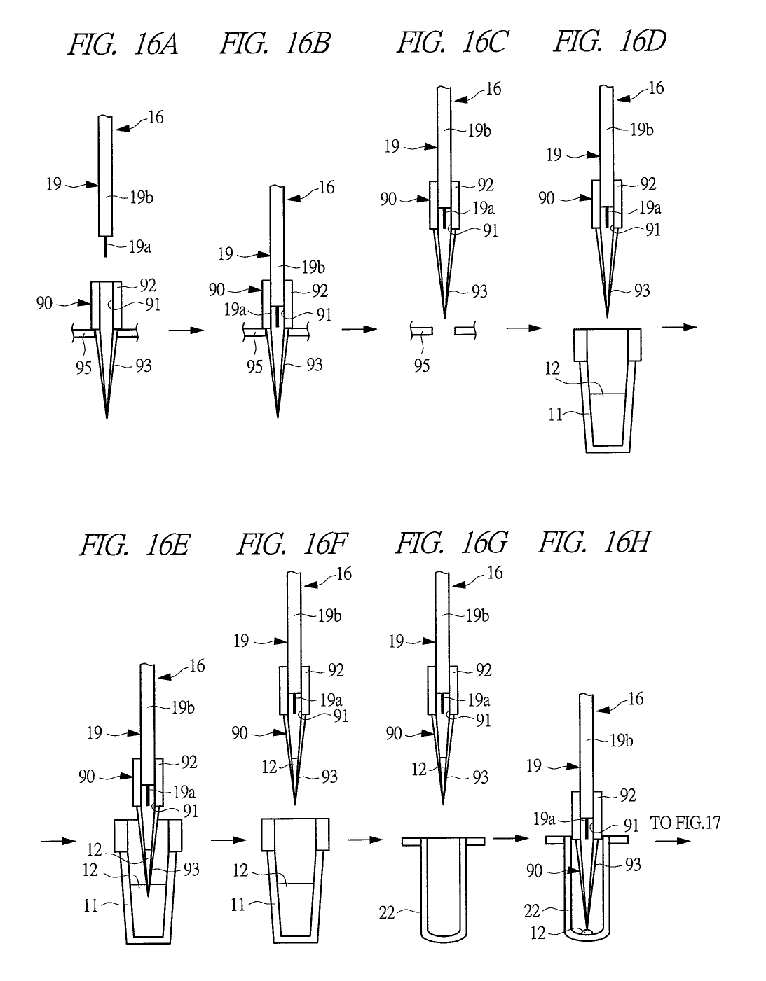

FIG. 14A is a main-part side view showing a main part of the sample dispensing probe in the case in which it is used with cleaning, and FIG. 14B is a main-part side view showing a main part of the sample dispensing probe in the case in which the disposable chip is used. FIGS. 15A to 15H are explanatory drawings for describing the flow of the operation of the sample dispensing probe in the case in which it is used with cleaning, and FIGS. 16A to 1611 and FIGS. 17A to 17C are explanatory drawings for describing the flow of the operation of the sample dispensing probe in the case in which the disposable chip is used. In these drawings, all elements are shown by solid lines for the convenience of understanding.

As shown in FIG. 14A, the sample dispensing probe 19 is provided with a probe main body 19a exposed at the distal end and a probe guard 19b protecting the probe main body and is configured to suck and discharge a sample by the probe main body 19a when it is used with cleaning.

As shown in FIG. 14B, the disposable chip 90 can be detachably attached to the probe guard 19b. The disposable chip 90 is provided with a probe inserting part 91 formed into a slit shape having the same diameter as the diameter of the probe guard 19b, a probe retaining part 92 formed to be thick at both ends of the probe inserting part 91, and a sample retaining part 93 extending so as to be tapered from the probe inserting part 91 to the distal end.

Since the probe inserting part 91 is formed to have the same diameter as the diameter of the probe guard 19b and the probe retaining part 92 abuts the both ends of the probe guard 19b, the disposable chip 90 can be attached without being fallen off from the probe guard 19b. In addition, since the probe retaining part 92 is formed to be thick, the disposable chip can be easily detached by pushing the part from the base end thereof toward the distal end.

As shown in FIG. 15A, when dispensing a sample 12 in the case of the use with cleaning, first, the sample dispensing probe 19 is moved to above the sample container 11 containing the sample 12. After the sample dispensing probe 19 is moved, as shown in FIG. 15B, the sample dispensing probe 19 is moved down, and the probe main body 19a enters and sucks the sample 12 in the sample container 11. After the sample is sucked, as shown in FIG. 15C, the sample dispensing probe 19 is moved up.

After the sample dispensing probe 19 is moved up, as shown in FIG. 15D, the sample dispensing probe 19 is moved to above the fixed container 21 in the example shown in the drawing. After the sample dispensing probe 19 is moved, as shown in FIG. 15E, the sample dispensing probe 19 is moved down, enters the fixed container 21, and discharges the sample 12. After the sample is discharged, as shown in FIG. 15F, the sample dispensing probe 19 is moved up.

After the sample dispensing probe 19 is moved up, as shown in FIG. 15G, the probe is moved to a probe cleaning mechanism 85. After the sample dispensing probe 19 is moved, as shown in FIG. 15H, cleaning liquid is discharged from a cleaning-liquid discharging part 86 of the probe cleaning mechanism 85, and the probe main body 19a is cleaned. Then, after the cleaning, the sample dispensing probe 19 is moved again to above the sample container 11, and the same operations are repeated. The probe cleaning mechanism 85 is present in the automatic analysis apparatus 1 shown in FIG. 1 to FIG. 6, but the mechanism is omitted in these drawings.

Also, as shown in FIG. 16A, when dispensing the sample 12 in the case of using the disposable chip 90, first, the sample dispensing probe 19 is moved to above the disposable chip 90 placed on a chip supplying board 95. After the sample dispensing probe 19 is moved, as shown in FIG. 16B, the sample dispensing probe 19 is moved down, and the disposable chip 90 is attached thereto. After the disposable chip 90 is attached, as shown in FIG. 16C, the sample dispensing probe 19 is moved up.

After the sample dispensing probe 19 is moved up, as shown in FIGS. 16D to 16F, the sample dispensing probe 19 is moved, is moved down, sucks the sample 12, and is moved up in the same manner as the case shown in FIGS. 15A to 15C in which the probe is used with cleaning. After the sample dispensing probe 19 is moved up in FIG. 16F, as shown in FIG. 16G, the sample dispensing probe 19 is moved to above the disposable container 22 in the example shown in the drawing. After the sample dispensing probe 19 is moved, as shown in FIG. 16H, the sample dispensing probe 19 is moved down and discharges the sample 12 into the disposable container 22.

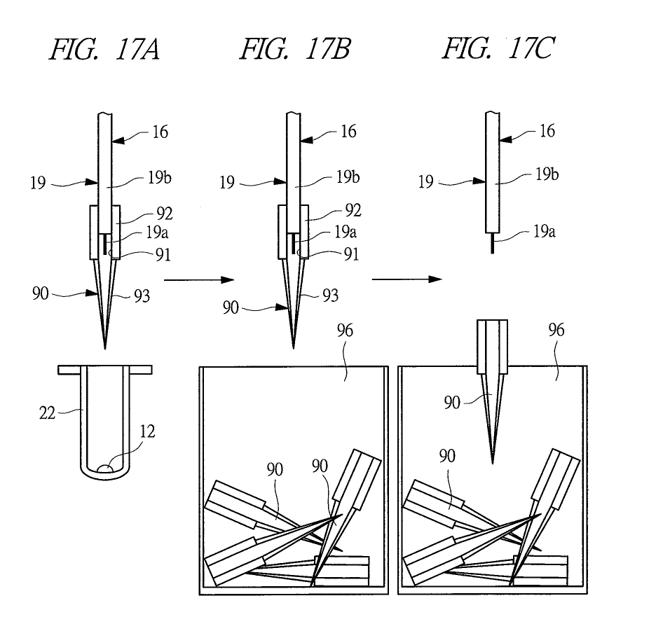

After the sample is discharged, as shown in FIG. 17A, the sample dispensing probe 19 is moved up. After the sample dispensing probe 19 is moved up, as shown in FIG. 17B, the sample dispensing probe 19 is moved to above a disposable chip discarding unit 96. After the sample dispensing probe 19 is moved, as shown in FIG. 17C, the disposable chip 90 is discarded into the disposable chip discarding unit 96. Then, after the disposable chip 90 is discarded, the sample dispensing probe 19 is moved again to above the chip supplying board 95, and the same operations are repeated. The chip supplying board 95 and the disposable chip discarding unit 96 are present in the automatic analysis apparatus 1 shown in FIG. 1 to FIG. 6, but the board and the unit are omitted in these drawings.

From the viewpoint of both the analysis accuracy and the cost, it is preferred that the disposable chip 90 is used in a highly-sensitive analysis such as an immunoserological test and the probe main body 19a of the sample dispensing probe 19 is cleaned and used in an analysis that does not require high sensitivity such as a biochemical test, a blood coagulation test and an electrolyte test.

A reagent probe of the reagent arm of the reagent dispensing mechanism may be similarly configured to be able to use the disposable chip.

As described above, in the automatic analysis apparatus 1 of the present invention, the pretreatment and reactions of the samples are carried out on the common disk 20, and the fixed containers 21 and the disposable containers 22 are disposed on the common disk 20. Therefore, a plurality of items can be tested with high accuracy and at low cost without increasing the size of the apparatus.

In other words, since the common disk 20 which is a multi-purpose disk capable of carrying out the pretreatment and reactions of the samples is provided, the apparatus can be downsized. In addition, when the fixed containers 21 are used in the case in which the containers are required to have high surface accuracy like in the case of colorimetric analysis and the disposable containers 22 are used in the case in which there is a high degree of necessity to prevent contamination among samples, highly accurate analysis can be carried out in any of test items while achieving the downsizing of the apparatus. Furthermore, when the fixed containers 21 are used in the case in which the containers are required to have high surface accuracy, since the disposable containers 22 are not required to be processed for improving surface accuracy, the cost of the disposable containers 22 can be reduced.

Moreover, in the automatic analysis apparatus 1 of the present invention, since the fixed containers 21 and the disposable containers 22 are regularly disposed on the common disk 20, arbitrary settings such as those described below can be implemented in accordance with requested contents.

(1) While biochemical tests are carried out in the fixed containers 21, the rates and the number of containers for carrying out pretreatment of the biochemical tests and flow tests are determined in accordance with the number of the requested items in the disposable containers 22. (2) In accordance with the quantity of the number of requested items of flow tests, the disposed rate of the disposable containers 22 is increased/decreased.

In other words, if the fixed containers and the disposable containers are irregularly disposed, it is difficult to synchronize the timing of the rotation cycle of the disk and the operations of the dispensing mechanisms, and therefore, it is difficult to carry out the control corresponding to a plurality of test items. On the other hand, since the fixed containers 21 and the disposable containers 22 are regularly disposed on the common disk 20, the rotation cycle of the common disk and the operations of the dispensing mechanisms can be easily controlled, and arbitrary settings can be implemented. Moreover, by virtue of this regular disposition, the control for the cleaning in the container cleaning mechanism 80 can also be facilitated.

Furthermore, in the automatic analysis apparatus 1 of the present invention, the sample dispensing mechanism 15 is capable of approaching an arbitrary container and dispensing a sample thereat. Therefore, by dispensing a sample for retest into the disposable container 22 in advance, re-sampling can be preferentially carried out for the analysis when the retest is required.

More specifically, since the rotation of a pretreatment disk has cycles, if the movement of a sample dispensing mechanism is restricted, re-sampling can be carried out only at the timing when the pretreatment disk comes close. Also, if the rotation cycle of the pretreatment disk is changed in accordance with the movement of the sample dispensing mechanism, it becomes difficult to control the analysis timing in a photometric mechanism. On the other hand, since the sample dispensing mechanism 15 can be moved without any restriction to an arbitrary container on the common disk 20, such preferential re-sampling can be carried out.

Furthermore, in the automatic analysis apparatus 1 of the present invention, the sample dispensing probe 19 of the sample dispensing arm 16 of the sample dispensing mechanism 15 is configured to allow both the use with cleaning and the use of the disposable chip 90. Therefore, a sample dispensing mechanism dedicated to the disposable chips is not required to be additionally provided, and the apparatus can be further downsized.

### INDUSTRIAL APPLICABILITY

The present invention can be utilized in an automatic analysis apparatus that automatically analyzes components of blood and others.

1. An automatic analysis apparatus comprising:
containers for carrying out a reaction between a sample and a reagent and/or pretreatment of the sample by pretreatment liquid;
a disk for the reaction and the pretreatment, on which the containers are disposed;
a sample dispensing mechanism for dispensing the sample into the container; and
a test mechanism for carrying out a plurality of items based on the reaction,
wherein, as the containers, a fixed container fixed to the disk for the reaction and the pretreatment and a disposable container detachably provided on the disk for the reaction and the pretreatment are disposed on the disk for the reaction and the pretreatment.

2. The automatic analysis apparatus according to claim 1,
wherein the fixed container and the disposable container are regularly disposed.

3. The automatic analysis apparatus according to claim 1,
wherein the fixed container and the disposable container are alternately disposed.

4. The automatic analysis apparatus according to claim 1,
wherein the sample dispensing mechanism is capable of approaching an arbitrary container among the containers of the disk for the reaction and the pretreatment and dispensing the sample.

5. The automatic analysis apparatus according to claim 1,
wherein the sample dispensing mechanism has (a) a sample dispensing arm, a first guide member guiding movement of the sample dispensing arm in a first direction and a second guide member guiding movement of the sample dispensing arm in a second direction orthogonal to the first direction or (b) a multi-joint arm provided with a rotation shaft at a center of the disk for the reaction and the pretreatment.

6. The automatic analysis apparatus according to claim 1,
wherein a sample for a retest is dispensed to the container in advance.

7. The automatic analysis apparatus according to claim 1,
wherein the sample dispensing mechanism has a sample dispensing probe which allows both a use with cleaning and a use of a disposable chip.
